(19) United States
(12) Patent Application Publication
(10) Pub. No.: US 2023/0250487 A1
(43) Pub. Date: Aug. 10, 2023

(54) TRANSMEMBRANE SENSORS AND MOLECULAR AMPLIFIERS FOR LYSIS-FREE DETECTION OF INTRACELLULAR TARGETS

(71) Applicant: ARIZONA BOARD OF REGENTS ON BEHALF OF ARIZONA STATE UNIVERSITY, Scottsdale, AZ (US)

(72) Inventors: Rizal HARIADI, Phoenix, AZ (US); Hao YAN, Gilbert, AZ (US); Swarup DEY, Middleton, WI (US)

(21) Appl. No.: 18/296,915

(22) Filed: Apr. 6, 2023

Related U.S. Application Data
(63) Continuation-in-part of application No. PCT/US2021/054861, filed on Oct. 13, 2021.
(60) Provisional application No. 63/091,113, filed on Oct. 13, 2020.

(51) Int. Cl. C12Q 1/6886; C12Q 1/682; C12Q 1/6881

(52) U.S. Cl. CPC C12Q 1/6886; C12Q 1/682; C12Q 1/6881; C12Q 1/6841; C12Q 2600/158; C12Q 2600/178

(57) ABSTRACT

Disclosed herein are methods and compositions comprising transmembrane nanosensors, and their use to identify, detect, label, and isolate cells. In some embodiments, the nanosensors may be used in conjunction with a hybridization chain reaction to amplify a signal, and to aid in the detection, identification, labeling or isolation of cells.

### CROSS-REFERENCE TO RELATED APPLICATIONS

This application is a continuation-in-part of International Application No. PCT/US2021/054861 with international filing date Oct. 13, 2021. This application claims the benefit of International Application No. PCT/US2021/054861 and U.S. Application No. 63/091,113, filed on Oct. 13, 2020, the content of each of which is incorporated herein by reference in its entirety.

### STATEMENT REGARDING FEDERALLY SPONSORED RESEARCH

None.

### BACKGROUND

The ability to sequence, genotype, and edit genomic information of cells, as well as to isolate cell subpopulation based on target biomarkers, are the hallmark for basic biological research and biomedical discovery. DNA sequencing enables researchers to determine the nucleic acid sequences down to the single-cell level.¹ Genome editing techniques, such as CRISPR^{2,3} and TALEN⁴, allow highly-specific insertions, deletions, and substitutions in target locations of the genome of live cells and afford the ability to correct inherent mutations in the genome that cause disease. Genotyping assays compare genomic fragments to assess the difference in gene variants associated with a disease or a phenotype of interest⁵ and to validate CRISPR gene-editing. The majority of existing quantitative analyses of expression of specific RNAs involves cell fixation or lysis of the cells to access the nucleic acid molecules; consequently, the cells are lost for downstream functional experiments. However, a genotyping technology that does not rely on lysis or fixation of cells or genetic manipulations for fast live-cell genotyping and RNA-based cell subpopulation sorting is still lacking.

In addition, immunologists or cell biologists who want to isolate cells whose biomarkers may be an mRNA that is expressed for functional studies of the cell type. As a result, all cell isolation techniques separate cells based on their surface biomarkers. Consequently, it is impossible to select a target cell subpopulation through surface protein-based methods if the mRNA biomarker of interest does not translate to surface biomarkers that can be captured by an antibody. Thus a need exists for detection of intracellular markers to use in cell selection methods such as Fluorescence-Activated Cell Sorting (FACS) and magnetic separation technology that do not require the cells to be lysed and leave the isolated cells in a functional state for further research. Therefore, a need exists for a method of detecting and sorting cells based on detection of intracellular markers.

### SUMMARY

The inventors provide a transmembrane nanosensor device. The device includes a lipid conjugated DNA tweezer comprising a hairpin loop complementary to a target polynucleotide trigger strand; a fluorophore; a quencher paired to the fluorophore; and an initiator sequence. When the hairpin loop is bound by the target polynucleotide trigger strand, the DNA tweezer transitions from a closed conformation to an open conformation and the quencher is separated from the fluorophore, and the fluorophore fluoresces. In the open conformation the initiator is exposed such that it can interact with a sensor.

A transmembrane nanosensor system is also provided in which the initiator interacts with the sensor to produce and optionally amplify a signal, and that can be used to detect and/or isolate the cells with an open conformation of the DNA tweezers. For example, the initiator may be paired with two hairpin nucleic acids to form a hybridization chain reaction and amplify the signal produced by the system.

Methods of using the transmembrane nanosensor and the system to detect or isolate cells expressing or carrying a particular nucleic acid are also provided. The cells may be used in downstream assays as described herein.

### BRIEF DESCRIPTION OF THE FIGURES

FIG. 1 shows both the design and characterization of a Transmembrane Nano Sensor (TraNS) capable of “sensing” a target nucleic acid. Panels A and B schematically depicts a tweezer-like DNA nanostructure for sensing target nucleic acid and which can switch from a “closed or “off” configuration (A) to an “open” or “on” configuration (B). The tweezer-like DNA nanostructure is asymmetric having at only one end a target-specific molecular beacon (green), fluorophore (orange), and quencher (black). The tweezer-like DNA nanostructure is modified with cholesterol for membrane insertion. The target specific molecular beacon can bind a specific target nucleic acid. Panel A shows that in the closed configuration, the quencher suppresses fluorophore emission. Panel B shows that upon binding of the target nucleic acid, such as RNA, by the molecular beacon, the DNA structure adopts the open configuration such that the fluorophore is no longer quenched and fluorescence increases. Panel C shows experimental data measuring fluorophore fluorescence in the absence and presence of a target miRNA. Panel D shows results from native PAGE analysis of the nanostructure in the absence and presence of a target miRNA. Panel E graphs the fluorescence per time representing the kinetics of a TraNS structure switching from closed to open (fluorescence) upon binding a target nucleic acid, which is either an RNA target (RNA) or DNA analog (DNA). Design and characterization of TraNS. (A-B) An asymmetric DNA nanostructure is modified with cholesterol for membrane insertion. A target-specific molecular beacon (MB) (green) is placed at the internal terminal. A quencher and a fluorophore are covalently conjugated in the MB in such a way that the quencher suppresses the emission of the fluorophore in the closed state. Upon sensing the target, the TraNS opens resulting in an enhanced fluorescence. (C-D) Efficient switching from closed to open TraNS observed by fluorescence spectra (C) and native PAGE (D). (E) Kinetics of TraNS switching upon binding to target RNA (dark blue) and target DNA (light blue).

FIGS. 2 shows the sensitivity, specificity, and kinetics of a TraNS probe in sensing membrane enclosed target DNA/RNA in cell-mimetic liposomes and patient derived exosomes. Panel A shows a schematic representation of a TraNS nanostructure that relies on both cholesterol modification to thermodynamically favor membrane insertion and a liposome encapuslated target nucleic acid in order to “sense” the target and emit a fluorescent signal (top right). The left top and bottom panels shows that no fluorescence is emitted in the absence of the target nucleic acid encapuslated in the liposome regardless of cholesterol modification. The bottom right panel shows that no fluorescene is emitted in the presence of the target nucleic encapsulated in the liposoome when there is no cholesterol modification of the TraNS nanostructure. Panel B shows experimental data equivalent to the schematics in the four panels in FIG. 2A using cell-mimetic 18:1 (Δ9-Cis) (DOPC) small unilamellar vesicles (SUVs) that encapsulated target DNA or random DNA. Membrane insertion of a TraNS nanostructure having a cholesterol modification that thermodynamically favors membrane insertion emitted a bulk fluorescence in the presence of SUV-enclosed target DNA. Negative controls with a TraNS nanostructure without cholesterol and SUVs with random DNA sequences lead to less than 0.1× fluorescence intensity, showing specificity to the SUV-enclosed target compared to the random DNA. Panels C and D show experimental data using cell-mimetic 16:0-18:1 PC (POPC) giant unilamellar vesicles (GUVs) that encapsulated target DNA or random DNA. Membrane insertion of a TraNS nanostructure having a cholesterol modification that thermodynamically favors membrane insertion emitted a bulk fluorescence in the presence of GUV-enclosed target DNA. Negative controls with a TraNS nanostructure without cholesterol resulted in dark GUVs (FIG. 2D), showing specificity to the GUV-enclosed target depends on membrane insertion. Sensitivity, specificity, kinetics of the TraNS device in sensing membrane enclosed target DNA/RNA in cell-mimetic liposomes and patient-derived exosomes. (A-B) TraNS-mediated signaling only occurs when insertion is thermodynamically-favored, and it finds target DNA. (C) Cholesterol-modified TraNS (green) bind to cell-mimetic GUVs. (D) TraNS without the cholesterol modification (green) stays in the media.

FIG. 3 shows different configurations for insertion of a TraNS DNA nanostructure into a membrane. Panels A and B show to possible orientations with the molecular beacon on the internal side of the membrane (A, “desired”) or on the external side of the membrane (B, “undesired”). Panel C shows a more asymmetrical TraNS DNA nanostructure having larger external portions than as in FIG. 1 (extending greater than 14 nanometers (nm)). Panel D shows a more asymmetrical TraNS DNA nanostructure having four larger external helices (extending greater than 14 nm) instead of two external helices. The more asymmetrical TraNS DNA nanostructures should further drive the thermodynamics favoring membrane insertion with the desired orientation having the large asymmetric portion on the extracellular side. TraNS designs with increasing asymmetry to investigate the effect of the complexity of the external terminal to the membrane orientation. (A and B) Initial design of TraNS with the desired (A) and undesired (B) orientations. (C) TraNS with larger external terminal. (D) TraNS with 4-helix external terminal.

FIG. 4 shows both diagrams and characterization of two metastable hairpins (H1 and H2) in the presence of an initiator molecule capable of linear DNA amplification via an enzyme-free hybridization chain reaction (HCR). Panel A shows a schematic diagram of an enzyme-free hybridization chain reaction (HCR) for linear amplification of a dsDNA from the two metastable hairpins H1 and H2. The initiator molecule (a*b*) triggers the opening of hairpin H1, which in turn leads to the opening of the second hairpin H2. H2 binds cb* sub-section of the first hairpin H1, and opens up, thus commencing a cyclical strand-displacement process. This continues until all the H1 and H2 hairpins in solution have been exhausted. Panel C shows experimental data demonstrating linear DNA amplification of two metastable H1 and H2 hairpins via an enzyme-free hybridization chain reaction via an exposed initiator domain DNA strand (center lane). In the absence of the initiator domain DNA, the H1 and H2 hairpins are stable (C: left lane). The presence of a ten-nucleotide segment that partially covers or “masks” the initiator domain DNA strand prevented HCR amplification (C: right lane). (A) Enzyme-free HCR on introduction of metastable hairpins resulting in a long dsDNA. (B-C) Schematic and agarose gel electrophoresis data shows that partially-covered initiator strand fails to trigger HCR.

FIG. 5 shows a schematic diagram of an allosteric TraNS probe providing HCR amplification. FIG. 5 depicts a tweezer-like DNA nanostructure for sensing a target nucleic acid and which can switch from a relaxed “closed or “off” configuration (C) to an “open” or “on” configuration (D) when tension exposes a cryptic initiator domain in the DNA nanostructure. The tweezer-like DNA nanostructure is asymmetric having at only one end a target-specific molecular beacon (green), fluorophore (orange), and quencher (black). The tweezer-like DNA nanostructure is modified with cholesterol for membrane insertion. The target specific molecule beacon can bind a specific target nucleic acid. Panels A and B shows a mechanosensitive multi-domain protein (e.g. vinculin) exposing a cryptic site by allostery as an analogy of the TraNS probe’s ability to provide a hybridization chain reaction (HCR) initiator upon target binding. Panel C shows that in the closed configuration, the cryptic initiator domain is inaccessible (like the cryptic site in vinculin (A)). Panel D shows that upon binding of the target RNA by the molecule beacon, the DNA structure adopts the open configuration such that the fluorophore is longer quenched and fluorescence increases and tension on the nanostructure exposes the cryptic initiator domain (similar to allosteric binding and tension exposing a cryptic site in a mechanosensitive multi-domain protein like vinculin (B)). Panel E shows that exposure of an extracellular cryptic initiator domain in the DNA nanostructure allows access for a HCR in the extracellular environment that provides signal amplification via the addition of multiple copies of the fluorophore. Biologically-inspired allosteric transmembrane sensor with HCR amplification reaction. (A-B) Mechanical tension exposed the cryptic site of a mechanosensitive proteins, such as Vinculin. (C-E) Binding to target RNA in the cytosol triggers the unlocking of initiator in the extracellular environment. The exposed initiator domains triggers HCR.

FIG. 6 shows a schematic diagram of applications of TraNS for live cell genotyping and sorting. In this application, TraNS probes are used for FACS-based sorting of live cells into target nucleic acid positive and negative subpopulations. The TraNS probe signal may be amplified. A red and a green laser is used to detect two different molecular beacons specific to two different target RNA sequences. Sorting living cells using RNA specific TraNS probes.

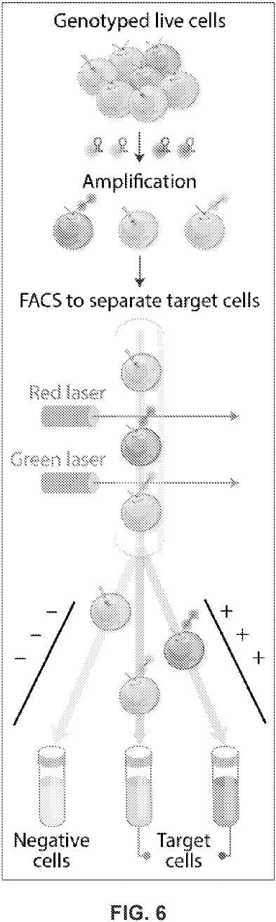

FIG. 7 shows a schematic diagram of applications of TraNS for live cell genotyping, sorting, and removal of the probe for further downstream uses of the sorted cells. In this application, a TraNS probe(s) is used for sorting living cells based on the presence of a target RNA marker(s). The TraNS probe signal may be amplified. Then for downstream assays and applications involving the sorted cells, the TraNS nanostructures may be degraded in the living cells by enzymatic sensor removal, such as via contacting the labeled cells with a non-specific exonuclease or DNA endonuclease. End-point genomic approaches of genotyping cells require cell lysis resulting in dead cells, which prevents using such genotyped cells in downstream assays and applications.

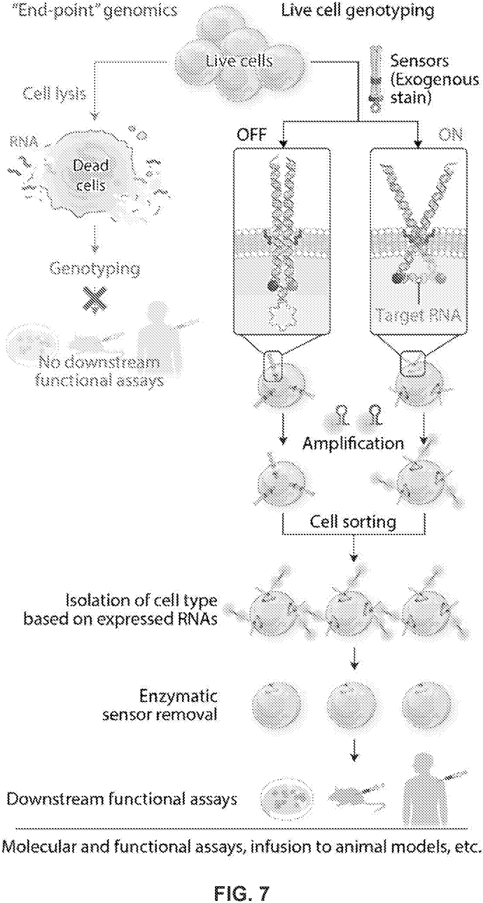

### DETAILED DESCRIPTION

Provided herein are reconfigurable transmembrane DNA nanosensors for an enzyme-free live-cell genotyping technique that will be specific for target cytosolic nucleic acid molecules such as RNA, and will enable researchers to use the genotyped cells for downstream applications. Currently, researchers isolate target cell subpopulations based on the surface protein antigens primarily due to the challenges of (i) detecting target intracellular nucleic acid molecules in the cytoplasm of live cells without cell lysis and (ii) transducing the information to the cell surface where the sensor reconfiguration can be amplified and the target cells can be “grabbed” for cell isolation. We propose a novel solution to these engineering challenges by using programmable oligonucleotide-based membrane-spanning sensors. We will augment the live-cell genotyping approach with enzyme free, isothermal amplification strategies, such as Hybridization Chain Reactions (HCR), to increase the signal for FACS and to serve as a “handle” for downstream cell isolation. Our live cell genotyping package will not only allow researchers to “sense” the nucleic acid molecules in cytosol, but also enable researchers to use the sub-population of target genotyped cells for downstream functional assays. To demonstrate the simplicity and evaluate the signal-to-noise ratio of our proposed approach, we will apply our technology to detect the expressed mRNA of GFP expressing HEK293 stable cell lines. We will further use the genotyped cells for cell isolation using FACS and magnetic cell separation based techniques. Collectively, these technologies will enable researchers to ask a diverse set of questions about the biology of cell types with different expressed RNA markers that are impossible to isolate with surface-marker-based cell separation techniques.

In some aspects, provided herein is a transmembrane nanosensor. In some embodiments, the transmembrane nanosensor includes a lipid conjugated DNA tweezer, a fluorophore, and a quencher suitably paired with the fluorophore. When the transmembrane nanosensor is in its un-bound state, the DNA tweezer is closed and the quencher and fluorophore are adjacent. When the target polynucleotide (i.e., the trigger strand) is bound to the hairpin loop, the DNA tweezer is in an open conformation, the quencher is separated from the fluorophore, and fluorescence from the fluorophore can be measured and quantitated.

Suitable quencher fluorophore pairs are known and described in the art. In some embodiments, the quencher-fluorophore pair are suitable for fluorescence resonance energy transfer (FRET).

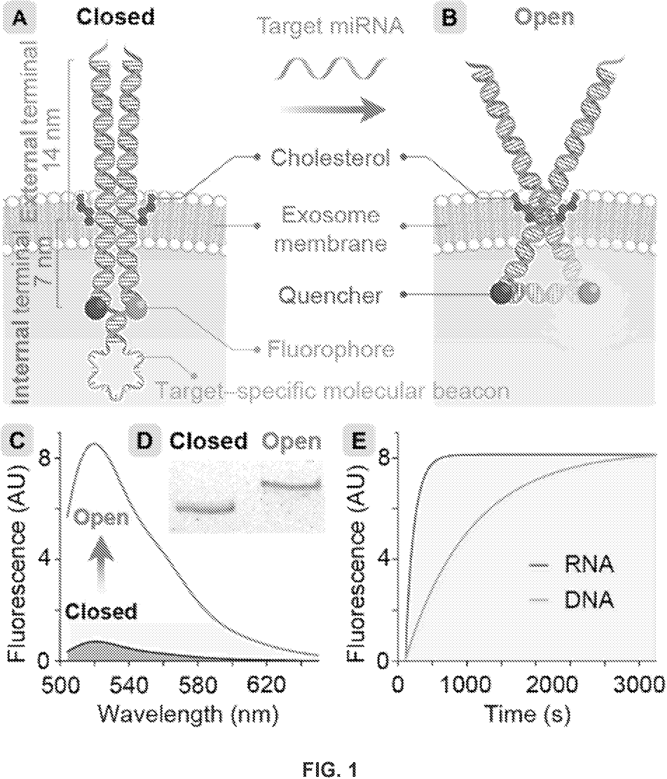

As used herein, “DNA tweezer” and “DNA nano-tweezer” are used interchangeably and refer to a nanoscale structure including a hairpin with a single-stranded loop and a first arm and a second arm linked by a crossover hinge wherein the distance between the tip of the first arm and the tip of the second arm is reversibly or irreversibly controlled by binding and release of a trigger strand to the single-stranded loop of the hairpin. In embodiments described herein, the trigger strand is external to the DNA nano-tweezer and is a target polynucleotide of interest. It will be readily understood by one of ordinary skill in the art that the flexibility and size of the DNA nano-tweezer may be manipulated by changing the size and sequences of DNA used in constructing the DNA nano-tweezer. In some embodiments, the first arm and second arm are double-crossover tile arms. In some embodiments, a more ridged multi-helix origami assembly may be utilized. One embodiment of a DNA nano-tweezer in both the closed and open conformation is depicted in Exhibit A, FIG. 1A. Conventional DNA nano-tweezer structures are known in the art. See for example Liu et al. (“A DNA tweezer-actuated enzyme nanoreactor,” Nature Communications, 2013, 4:2127); Zhou et al. (“Reversible regulation of protein binding affinity by a DNA machine,” J. Am. Chem. Soc., 2012, 134(3), 1416-1418); and U.S. Application No. 16/653,253.

As used herein, “closed conformation” refers to the conformation of the DNA nano-tweezer wherein the hairpin loop is free and unbound by a trigger strand. In the closed conformation, the distance between the tip of the first arm and the tip of the second arm is about 4 nm (e.g., 3, 4, 5, or 6 nm). In some embodiments, the distance between the tip of the first arm and the tip of the second arm in the closed conformation is between about 3 nm and about 18 nm, between 3 nm and 16 nm, between 4 nm and 14 nm, or between 4 nm and about 10 nm. In some embodiments, the distance between the tip of the first arm and the tip of the second arm is less than 18 nm, less than 17 nm, less than 16 nm, less than 15 nm, less than 14 nm, less than 13 nm, less than 12 nm, less than 11 nm, less than 10 nm, less than 9 nm, less than 8 nm, less than 7 nm, less than 6 nm, less than 5 nm, less than 4 nm, less than 2 nm, or less than 1 nm. In the closed conformation, the quencher quenches the detectable label of a label/quencher pair. In the closed conformation, the HCR initiator is masked, and cannot initiate a HCR.

As used herein, “open conformation” refers to the conformation of the DNA nano-tweezer wherein the trigger strand is bound to the hairpin loop. In the open conformation, the distance between the tip of the first arm and the tip of the second arm is about 16 nm (e.g., 12 nm, 13 nm, 14 nm, 15 nm, 16 nm, 17 nm, 18 nm, 19 nm, or 20 nm). In some embodiments, the distance between the tip of the first arm and the tip of the second arm in the open conformation is between about 12 nm and about 20 nm, between about 13 nm and about 19 nm, between about 14 nm and about 18 nm, or between about 15 nm and about 17 nm. In some embodiments, the distance between the tip of the first arm and the tip of the second arm in the open conformation is at least 12 nm, at least 13 nm, at least 14 nm, at least 15 nm, at least 16 nm, at least 17 nm, at least 18 nm, at least 17 nm, at least 20 nm, at least 30 nm, or at least 40 nm. In the open conformation, the quencher and the detectable label of a label/quencher pair are separated sufficiently to eliminate quenching. In the open conformation, the HCR initiator is unmasked, and is available to HCR reaction components such as HCR hairpins, to initiate a HCR.

In various embodiments of the DNA nano-tweezers described herein, binding of the trigger loop to the hairpin loop results in an increase in the distance between the tip of the first arm and the tip of the second arm and increases the distance between the fluorophore and the quencher. The increase in distance between the fluorophore and the quencher may be an increase of about 4 nm, 6 nm, 8 nm, 10 nm, 11 nm, 12 nm, 13 nm, 14 nm, 16 nm, or more.

As used herein, “trigger strand” refers to a nucleic acid oligonucleotide that is complementary to and binds to the hairpin loop of the DNA nano-tweezer to initiate a conformation change in the DNA nano-tweezer from the closed conformation to the open conformation. In the embodiments described herein, the trigger strand is target polynucleotide of interest. In some embodiments, the trigger strand may be between about 14 bases and about 40 bases (e.g., 15 to 35 bases, 18 to 30 bases, 20 bases to 28 bases) in length. In some embodiments, the trigger strand is about 21 bases in length (e.g., 15 bases, 16 bases, 17 bases, 18 bases, 19 bases, 20 bases, 21 bases, 22 bases, 23 bases, 24 bases, or 25 bases).

As used herein, a “hybridization chain reaction” (HCR) is an enzyme-free amplification reaction that extends a DNA molecule via incorporation of two or more metastable DNA hairpin stem-loops via hybridization of complementary sequences between the stem-loops and the DNA molecule.

As used herein, an “HCR initiator molecule” or “initiator molecule” is a single-stranded DNA comprising both (1) a polynucleotide sequence complementary to one of the ends (such as the 5′ end) of a first metastable DNA hairpin and (2) a polynucleotide sequence complementary to the stem region adjacent to the one of the ends, e.g., the 5′ stem region, of the first metastable DNA hairpin.

As used herein, a “HCR initiator domain,” “initiator sequence,” or “initiator domain” refers to a nucleic acid which binds to an HCR initiator molecule via hybridization of complementary sequences and initiates a linear HCR amplification of a DNA molecule comprising the HCR initiator domain.

As used herein, a “3′ polynucleotide” refers to a DNA domain at the 3′ end of a nucleic acid structure, for example, of a hairpin stem-loop. With respect to a hairpin loop, a 3′ polynucleotide would be positioned at the 3′ end of the hairpin stem loop and is not part of the stem region but rather extends beyond the stem region at the 3′ end. Likewise, a 5′ polynucleotide refers to a DNA domain at the 5′ end of a nucleic acid structure, for example, of a hairpin stem-loop. With respect to a hairpin loop, a 5′ polynucleotide would be positioned at the 5′ end of the hairpin stem loop and is not part of the stem region but rather extends beyond the stem region at the 5′ end.

As used herein, the term “nucleotide” or “nucleotide moiety” refers to a sub-unit of a nucleic acid (whether DNA or RNA or analogue thereof) which includes a phosphate group, a sugar group and a heterocyclic base, as well as analogs of such sub-units. A “nucleotide monomer” refers to a molecule which is not incorporated in a larger oligo- or poly-nucleotide chain and which corresponds to a single nucleotide sub-unit. In some cases, other groups (e.g., protecting groups) can be attached to any component(s) of a nucleotide or nucleotide monomer.

A “nucleoside” or “nucleoside moiety” refers to a nucleic acid subunit including a sugar group and a heterocyclic base, as well as analogs of such sub-units. Other groups (e.g., protecting groups) can be attached to any component(s) of a nucleoside. A “nucleoside residue” refers to a molecule having a sugar group and a nitrogen containing base (as in a nucleoside) as a portion of a larger molecule, such as in a polynucleotide, oligonucleotide, or nucleoside phosphoramidite.

As used herein, the terms “nucleic acid polymer,” “nucleic acids,” of “polynucleotide” refer to polymers comprising nucleotides or nucleotide analogs joined together through backbone linkages such as but not limited to phosphodiester bonds. Nucleic acids include deoxyribonucleic acids (DNA) and ribonucleic acids (RNA) such as messenger RNA (mRNA), transfer RNA (tRNA), as well as other hybridizing nucleic-acid-like molecules such as those with substituted backbones, e.g., peptide nucleic acids (PNAs) or other nucleic acids comprising modified bases and sugars. In some cases, the target nucleic acid is a double stranded DNA. In some cases, the target nucleic acid is cell-free DNA (cfDNA). However, the methods of the invention are not limited to double stranded DNA because other nucleic acid molecules, such as a single stranded DNA or RNA can be turned into double stranded DNA by one of skill in the arts using known methods. Suitable double stranded target DNA may be a genomic DNA or a cDNA.

Nucleic acids and/or other moieties of the invention may be isolated. As used herein, “isolated” means to separate from at least some of the components with which it is usually associated whether it is derived from a naturally occurring source or made synthetically, in whole or in part.

Nucleic acids and/or other moieties of the invention may be purified. As used herein, purified means separate from the majority of other compounds or entities. A compound or moiety may be partially purified or substantially purified. Purity may be denoted by a weight by weight measure and may be determined using a variety of analytical techniques such as but not limited to mass spectrometry, HPLC, etc.

The terms “detect” or “detection” as used herein indicate the determination of the existence, presence or fact of a target or signal in a limited portion of space, including but not limited to a sample, a reaction mixture, a molecular complex and a substrate including a platform and an array. Detection is “quantitative” when it refers, relates to, or involves the measurement of quantity or amount of the target or signal (also referred as quantitation), which includes but is not limited to any analysis designed to determine the amounts or proportions of the target or signal. Detection is “qualitative” when it refers, relates to, or involves identification of a quality or kind of the target or signal in terms of relative abundance to another target or signal, which is not quantified. An “optical detection” indicates detection performed through visually detectable signals: fluorescence, spectra, or images from a target of interest or a probe attached to the target.

For the purposes of this disclosure, the term “target” refers to a nucleic acid molecule or polynucleotide that is the intended nucleic acid molecule to be detected by a transmembrane nanosensor as described herein, i.e,. bound by the hairpin stem loop polynucleotide sequence which is complementary to the target polynucleotide. The target may be an mRNA or intracellular RNA molecule such as an miRNA, iRNA, or other cellular nucleic acid including DNA.

The transmembrane nanosensor may further include an initiator polynucleotide sequence for initiating a hybridization chain reaction. In nanosensors comprising an initiator sequence, the initiator sequence is masked when the nanosensor is in a closed conformation, and is unmasked when the nanosensor is in an open conformation. As used herein, the term “masked” with respect to the initiator means that the initiator cannot be bound by sensor molecules, e.g., metastable hairpins configured for HCR with the initiator polynucleotide sequence. Conversely, an unmasked initiator is accessible to sensor molecules such as HCR hairpins, and can initiate HCR.

As used herein, the term “sensor molecule” refers to a molecule that can hybridize to an unmasked initiator molecule. Sensor molecules may include detectable labels and/or quencher/label pairs. In some embodiments, a sensor molecule comprises a hairpin configured for HCR.

In an exemplary embodiment, HCR metastable hairpins comprise one or more fluorescent markers. In some embodiments, the HCR metastable hairpins further comprise quenchers, such that the fluorescent marker is quenched when the hairpin is unbound (e.g., in hairpin form), and the fluorophore is unquenched when the hairpin is open (e.g., bound to its hybridization partner).

In an exemplary, non-limiting embodiment, two additional hairpins are used, and are labeled with a fluorescent marker and a quencher such that in the hairpin configuration the fluorophore is paired with the quencher. For example, in some embodiments, the initiator sequence is complementary to the 5′ end and 5′ stem of the first hairpin and is the same sequence as the loop and the 3′ stem of a second HCR hairpin, and wherein the first hairpin loop and 3′ end of the stem are complementary to the 5′end and the 5′ stem of the second hairpin. The initiator sequence may only be exposed when the DNA tweezer is in the open conformation. When the DNA tweezer is in the open conformation and the two hairpins are included in the system described herein the initiator sequence binds to the first hairpin and via strand displacement of the stem by binding to the initiator, thereby separating the fluorophore from the quencher of the first hairpin. The loop and 3′ stem of the first hairpin then binds to the 5′ tail and stem of the second hairpin via strand displacement and the fluorophore of the second hairpin is separated from the second quencher. This process can continue to repeat itself as the single-stranded loop and 3′ stem of the second hairpin are complementary to the 5′ tail and stem of the first hairpin. This allows for amplification of the fluorescence signal from the two hairpins. This signal can them be amplified enough for use in fluorescent activated cell sorting applications or other cell separation methods. It is understood that the reverse orientation of hairpins and initiator molecule is also embodied herein.

Exemplary method of detecting an HCR product are well known in the art and include without limitation, detecting a detectable signal from the HCR product, e.g., a fluorescent signal, binding the HCR product to one or more labeled probes and detecting the bound, labeled probes, capturing the HCR product to a solid support, isolating the nanosensors after HCR reaction, and performing gel electrophoresis, etc.

Methods of labeling a cell using the transmembrane nanosensor or the transmembrane nanosensor system provided herein are also provided. The methods include contacting the cell with the transmembrane nanosensor system; and measuring the fluorescence of the transmembrane nanosensor system and/or a HCR product, and/or isolating the cells demonstrating fluorescence and/or an HCR product. The isolated cells may be used in further functional assays. Methods of removing the transmembrane nanosensor from the treated cells are also available to those of skill in the art and as described in the Examples that follow.

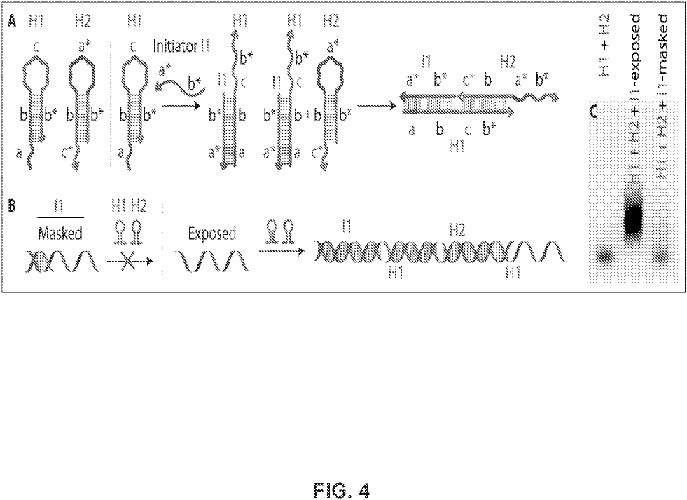

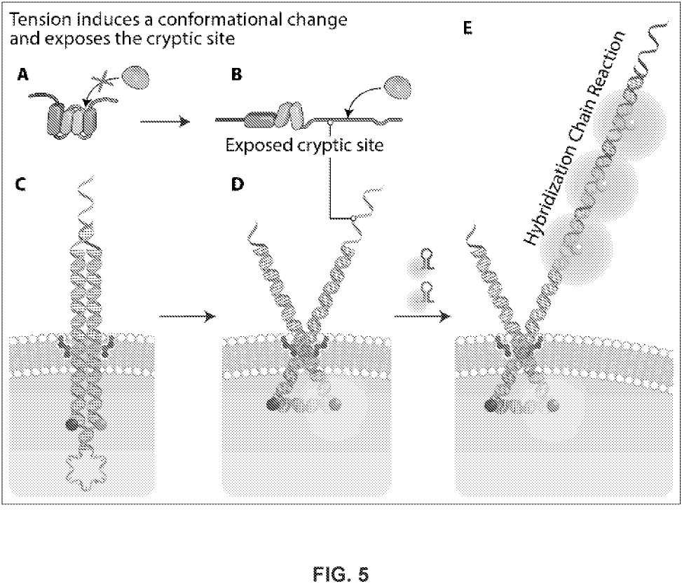

An exemplary embodiment is a transmembrane nanosensor comprising: (1) a lipid-conjugated DNA comprising a hairpin stem-loop comprising: (a) a loop comprising a polynucleotide sequence which is complementary to a target polynucleotide, (b) a stem comprising complementary 5′ and 3′ domains, (c) a fluorophore, (d) a quencher paired to the fluorophore, and (e) a hybridization chain reaction (HCR) initiator domain linked to the end of either the 5′ or 3′ domain of the stem. Wherein the nanosensor, upon the hairpin stem-loop binding to the target polynucleotide, the nanosensor transitions from a closed conformation to an open conformation exposing the HCR initiator domain and allowing for fluorescence without quenching. See e.g., FIGS. 4 and 5.

In some embodiments, components for an HCR reaction are provided, and include without limitation, metastable hairpins, at least one of which hybridizes to the initiator molecule to initiate the HCR reaction. In some embodiments the metastable HCR hairpins comprise a detectable label, and/or a quencher/label combination such that the detectable label is quenched until the hairpin is opened, e.g., by binding to its complementary initiator sequence on the transmembrane nanosensor in the open conformation. Thus, in some embodiments, a metastable hairpin (MS hairpin) comprises: a hairpin stem-loop comprising: (a) a polynucleotide which is complementary to the HCR initiator domain, (b) a stem comprising complementary 5′ and 3′ domains wherein the 5′ domain or the 3′ domain and at least a portion of the loop of the MS hairpin is complementary to the initiator of the lipid-conjugated hairpin. In some embodiments, the MS hairpins comprise a detectable label. In some embodiments, the MS hairpins comprise a detectable label/quencher pair.

In some exemplary embodiments, the lipid-conjugated hairpin spans a lipid bilayer or is integrated into a lipid bilayer. In some exemplary embodiments, the lipid bilayer is a cellular outer membrane or episome.

In some exemplary embodiments, the target polynucleotide is or comprises an RNA or a DNA polynucleotide. In some exemplary embodiments, the target polynucleotide is or comprises a messenger RNA (mRNA) or microRNA (miRNA).

In some exemplary embodiments, the lipid conjugated to the transmembrane nanosensor comprises or consists of a cholesterol molecule.

In some exemplary embodiments, upon the open conformation exposing the HCR initiator domain, the HCR initiator domain is bound by an

An exemplary embodiment comprises a method of labeling a cells that comprise a target polynucleotide, the method comprising: (a) contacting a population of cells with a nanosensor or nanosensor system described herein; and (b) measuring fluorescence of the transmembrane nanosensors, whereby fluorescence of the transmembrane nanosensor indicates the presence of a cell comprising the target polynucleotide. In some exemplary embodiments, the method comprises separating a fluorescent nanosensor-labeled cell detected by the transmembrane nanosensor away from another cell, such as a non-fluorescent cell or cell lacking fluorescence indicative of nanosensor detection of the target polynucleotide. In some exemplary embodiments, the cell is derived from a subject. In some exemplary embodiments, the cell is from a liquid biological sample from a subject. In some embodiments, the cell is from a solid biological sample from the subject, such as a biopsy sample. In some exemplary embodiments, the target polynucleotide is an miRNA, mRNA, or DNA biomarker specific to cancer. In some exemplary embodiments, the measuring comprises quantitating the fluorescence. In some exemplary embodiments, a cell comprising the polynucleotide of interest is labeled, identified and/or sorted via detection of HCR products. By way of example, in some embodiments, the nanosensor, when in the open conformation, exposes an HCR initiator sequence in addition to providing a fluorescent signal. In some embodiments, the HCR product is configured for cell isolation or sorting (e.g., by hybridization to a capture molecule linked to a solid support). In some embodiments, the HCR product comprises one or more detectable labels.

An exemplary embodiment comprises a method of diagnosing a disease in a subject, the method comprising: (a) contacting a cell derived from the subject with a nanosensor or nanosensor system described herein and (b) measuring fluorescence of a transmembrane nanosensor, whereby fluorescence of the transmembrane nanosensor indicates the presence of a target polynucleotide indicative of the disease in the subject. In some exemplary embodiments, the cell derived from the subject is from a liquid biological sample from the subject, or from a solid biological sample from the subject (e.g., such as a tumor biopsy sample). In some exemplary embodiments, the target polynucleotide is an RNA, miRNA, or DNA biomarker specific to cancer. In some exemplary embodiments, the measuring comprises quantitating the fluorescence. In some embodiments, the method of diagnosing comprises detecting an HCR products. By way of example, in some embodiments, the nanosensor, when in the open conformation, exposes (unmasks) an HCR initiator sequence in addition to providing a fluorescent signal. In some embodiments, the HCR product is configured for detection, e.g., comprises one or more detectable labels. In some embodiments, the HCR product is configured for cell isolation and/or sorting (e.g., by hybridization to a capture molecule linked to a solid support).

Additional exemplary embodiments of the disclosure are provided below, numbered 1-23.

1. A transmembrane nanosensor comprising (a) a lipid-conjugated DNA tweezer comprising a hairpin loop complementary to a target polynucleotide trigger strand; (b) a fluorophore; and (c) a quencher paired to the fluorophore (or a FRET pair); wherein upon hairpin loop binding a target polynucleotide trigger strand, the DNA tweezer transitions from a closed conformation to an open conformation resulting in a separation of the quencher from the fluorophore allowing the fluorophore to fluoresces without quenching.

2. A transmembrane nanosensor according to embodiment #1, wherein the lipid-conjugated DNA tweezer is integrated into a lipid bilayer.

3. A transmembrane nanosensor according to embodiment #2, wherein the lipid bilayer is a cellular membrane or exosome membrane.

4. A transmembrane nanosensor according to any one of embodiments #1-3, wherein the target polynucleotide trigger strand is or comprises an RNA or a DNA polynucleotide.

5. A transmembrane nanosensor according to embodiment #4, wherein the target polynucleotide trigger strand is a messenger RNA (mRNA) or a micro RNA (miRNA).

6. A transmembrane nanosensor according to any one of embodiments #1-5, wherein the lipid is or comprises a cholesterol molecule.

7. A transmembrane nanosensor according to any one of embodiments #1-8, comprising an initiator sequence or hybridization chain reaction (HCR) cyclical strand-displacement.

7-B. A transmembrane nanosensor according to embodiment #7, wherein the open conformation of the DNA tweezer exposes the initiator sequence.

8. A transmembrane nanosensor system, comprising the transmembrane nanosensor of any one of embodiments #7 and #7-B and a first hairpin nucleic acid, a second hairpin nucleic acids, wherein the first and second hairpin nucleic acids are labeled with a fluorescent marker and a quencher such that in the hairpin configuration the fluorophore is paired with the quencher, wherein the initiator sequence is complementary to the 5′ end and 5′ stem of the first hairpin and the same sequence as the loop and the 3′ stem of the second hairpin, and wherein the first hairpin loop and 3′ end of the stem are complementary to the 5′end and the 5′ stem of the second hairpin.

9. A transmembrane nanosensor system according to embodiment #8, wherein when the DNA tweezer transitions from a closed conformation to an open conformation the initiator is exposed and binds to the first hairpin and via strand displacement of the stem by binding to the initiator the fluorophore is separated from the quencher and the first hairpin binds to the second hairpin via strand displacement and the fluorophore of the second hairpin is separated from the second quencher.

10. A method of labeling a cell, the method comprising: (i) contacting the cell with the transmembrane nanosensor of any one of embodiments #1-7-B; and (ii) measuring or quantitating fluorescence of the transmembrane nanosensor, wherein fluorescence of the nanosensor indicates the presence of the target polynucleotide trigger in the cell.

11. A method of labeling a cell using the transmembrane nanosensor system of any one of embodiments #8-9, the method comprising: (i) contacting the cell with the transmembrane nanosensor system; and (ii) measuring or quantitating fluorescence of the transmembrane nanosensor system.

12. A method of according to embodiment #11, further comprising separating a fluorescent cell from another cell.

13. A method diagnosing a disease in a subject, the method comprising: (a) contacting an exosome or cell derived from the subject with a transmembrane nanosensor according to any one of embodiments #1-9; and (b) measuring fluorescence of the transmembrane nanosensor, whereby fluorescence of the transmembrane nanosensor indicates the presence of the target polynucleotide in the cell.

14. A method according to embodiments #13, wherein the exosomes or cells are from a liquid biological sample from the subject.

15. A method according to any one of embodiments #13-14, wherein the target polynucleotide trigger is an miRNA biomarker specific to cancer.

16. A method according to any one of embodiments #13-15, wherein the measuring comprises quantitating the fluorescence.

22.A nanosensor comprising: (i) a transmembrane nanosensor comprising a lipid-conjugated DNA comprising a first hairpin stem-loop comprising: a loop comprising a polynucleotide sequence which is complementary to a target polynucleotide, a stem comprising complementary 5′ and 3′ domains, a fluorophore, a quencher paired to the fluorophore, and an HCR initiator domain 5′ of the 5′ domain of the stem; and (ii) a second hairpin stem-loop comprising: a second loop comprising a polynucleotide which is complementary to the HCR initiator domain, a second stem comprising complementary second 5′ and second 3′ domains wherein the second 5′ domain is complementary to the 5′ domain of the first hairpin stem-loop and wherein the second 3′ domain is complementary to the 3′ domain of the first hairpin stem-loop, a second fluorophore, a second quencher paired to the second fluorophore, and a 3′ polynucleotide 3′ of the second 3′ domain of the second stem and complementary to the loop of the first hairpin stem-loop; wherein upon the first hairpin stem-loop binding to the target polynucleotide, the hairpin stem-loop transitions from a closed conformation to an open conformation exposing the HCR initiator domain and allowing for fluorescence the fluorophore without quenching. In some embodiments, the lipid-conjugated DNA spans a lipid bilayer. In some embodiments, the lipid bilayer is a cellular outer membrane or episome. In some embodiments, the target polynucleotide is an RNA, such as messenger RNA (mRNA) or microRNA (miRNA), or a DNA molecule. In some embodiments, the lipid comprises a cholesterol molecule. In some embodiments, the nanosensor comprises an initiator molecule comprising a linear DNA comprising: a 5′ end polynucleotide complementary to the 5′ domain of the first hairpin stem-loop and the second 5′ domain of the second hairpin stem-loop, and a 3′ end polynucleotide complementary to the loop of the second hairpin stem-loop and the HCR initiator domain; wherein upon the first hairpin stem-loop binding to the target polynucleotide, the hairpin stem-loop transitions from a closed conformation to an open conformation exposing the HCR initiator domain resulting in the initiator molecule binding the HCR initiator domain initiating an HCR amplification via strand displacement resulting in the first fluorophore separating away from the first quencher and subsequently resulting in strand displacement of the second hairpin stem-loop and the second fluorophore separating away from the second quencher.

23. Also disclosed herein are methods of labeling a cell. In some embodiments, the method comprises (a) contacting the cell with the transmembrane nanosensor of, for example, embodiment 22, measuring fluorescence of the transmembrane nanosensor and/or detecting a product of HCR; whereby fluorescence of the transmembrane nanosensor and/or detection of an HCR product is indicative of the target polynucleotide in the cell. In some embodiments, the method further comprises separating a fluorescent-labeled cell and/or HCR product expressing cell away from another cell.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

Designing transmembrane sensors for lysis-free intracellular RNA detection of live cells.

For lysis-free RNA sensing, one of the engineering challenges is inserting the hydrophilic transmembrane sensors through the hydrophobic lipid bilayer. Evolution has given rise to transmembrane protein sensors and protein signal transducers, such as G-protein coupled receptors (GPCRs). These membrane proteins relay information across the cell membranes²⁶. GPCRs consist of a reconfigurable²⁷, hydrophilic-hydrophobic-hydrophilic (Hi-Ho-Hi) molecular structure where the hydrophobic part is buried in the cell membranes while the two hydrophilic ends remain on the two sides of the bilayer²⁸. Using this design principle, we propose GPRC analog of transmembrane sensors to detect cell-enclosed target RNA biomarkers. We engineered a cholesterol-modified oligonucleotide-based structure that spontaneously inserts through lipid bilayer and dynamically reconfigures upon sensing a target RNA molecules.

### Preliminary Results

Design of the Transmembrane Nano Sensor (TraNS). We designed a tweezer-like DNA nanostructure that can switch from an OFF to an ON configuration upon sensing target DNA (FIGS. 1A-B). The nanodevice consists of a molecular beacon that can sense a specific target DNA. Upon binding the specific DNA target, the TraNS device switches to an ON configuration, which is detected by an increase in fluorescence in the fluorescence spectra (FIG. 1C). PAGE gel electrophoresis (FIG. 1D) and fluorescence experiments (FIG. 1E) confirm the efficient switching of TraNS. Binding of TraNS sensors with RNA molecules is faster than with target DNA analogs (FIG. 1E).

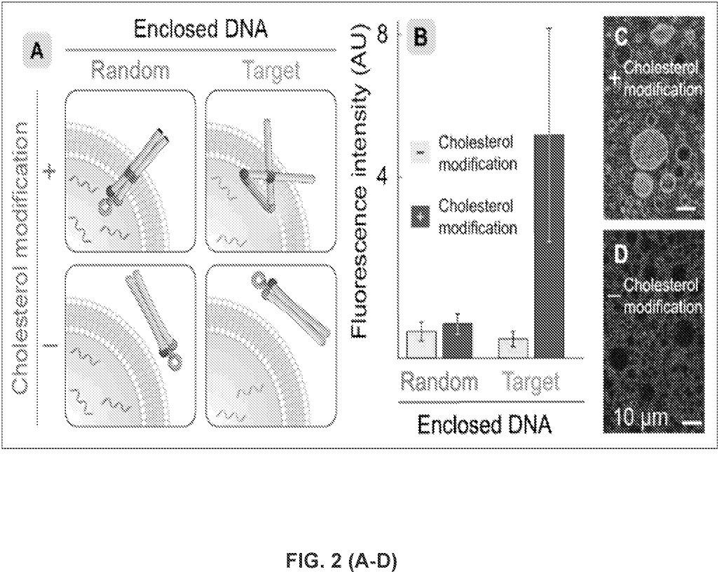

Specificity and sensitivity of TraNS device in cell-mimetic small and giant liposomes. To test the sensing performance of the TraNS, we prepared cell-mimetic 18:1 (Δ9-Cis) (DOPC) small unilamellar vesicles (SUVs; FIG. 2B) and 16:0-18:1 PC (POPC) giant unilamellar vesicles (GUVs; FIGS. 2C-D) encapsulating target DNA. Fluorescence spectra show that the cholesterol modified TraNS device selectively inserts through the membrane. Upon binding to the membrane-enclosed target DNA, there is an increase in bulk fluorescence (FIG. 2B) and the fluorescence of GUVs (FIG. 2C) due to the membrane insertion and opening of TraNS by target DNA molecules. Negative controls with TraNS without cholesterol and SUVs with random DNA sequences lead to <0.1× fluorescence intensity (FIG. 2B) and dark GUVs (FIG. 2D), showing that the sensing performance of TraNS is specific to the SUV-enclosed target.

Specific detection of membrane-enclosed RNA in extracellular vesicles. To test the performance of TraNS in biological systems, we engineered a specific TraNS for a lung cancer biomarker RNA (miR-21-5p)^{29,30} and tested on exosomes derived from (i) A549 human non-small cell lung cancer cell lines, and (ii) healthy donor serum. miR-21-5p has been found as a diagnostic and prognostic miRNA marker for several types of cancers, e.g. non-small cell lung cancer^{29,30}, pancreatic cancer³¹, diffuse large B-cell lymphoma^{32,33}, breast cancer³⁴, and colorectal cancer³⁵.

Detection of mRNA of reporter proteins in mammalian cells. Inducible HEK293 GFP stable cell line obtained from GenTarget Inc will be used for live-cell genotyping experiments. The codon optimized GFP is expressed under an inducible suCMV promoter. The cell line constantly expresses the repressor protein (tetR) which stops the GFP’s expression. The GFP expression only occur when the inducer (tetracycline) was added into the culture medium. Control experiments will be done with HEK-293 cells from AATC. We will sequence the HEK-293 control cells to confirm the absence of GFP mRNA in the control HEK293 and in the HEK293 GRP in the absence of tetracycline. To minimize any emission overlap with GFP, we will use molecular beacons with red flurophores, such as Cy5.

First, we will quantify the average number of TraNS in each HEK293 GFP cell by counting the number of membrane-bound TraNS with Oxford NanoImager single-molecule fluorescence microscope. HEK293 GFP cells will treated with Alexa 647-labeled TraNS and HEK293 GFP cells at varying concentration for 30 minutes in cell media (DMEM, L-glutamine, 10% FBS, MEM Non-essential amino acids, and antibiotics), followed by washing steps to remove the unbound TraNS. The fluorescence signal will be quantified with home-built Mathematica code using its Image Analysis package.

We will then use the concentrations of TraNS that corresponds 0-1000 TraNS per cell to detect GFP mRNA in live cells before and after the addition of Tetracycline. A 20-µm spherical cell with 1000 TraNS corresponds to an average of 1 sensor in 1 µm² of cell surface. Here, the molecular beacon domain of TraNS will have a fluorophore and a quencher. Upon sensing the mRNA of GFP, the TraNS switches from OFF to ON state resulting in an enhanced fluorescence. We will count the average number of ON TraNS per cell using Oxford NanoImager single molecule microscope at 0-24 hour time points at 37° C. and 5% CO₂. We will perform the assay using at least 3 replicates. As negative controls, we will use HEK293 without tetracycline and TraNS with random specificity. These negative control experiments will be used to establish the baseline fluorescence due to TraNS and cell autofluorescence.

Kinetics of GFP mRNA-TraNS interactions in cell. In preliminary experiments, bulk fluorescence traces of experiments using cell-mimetic SUVs with target DNA (FIG. 1E) show that the reactions reach 50% completion in under 30 mins and plateaus after ~3 hours. We will measure the kinetics of TraNS opening in HEK293 GFP cells using bulk fluorometer at 0-1000 TraNS/cell ratios in a Nanolog fluorometer (Horiba) with 645 nm excitation and emission window between 670-800 nm. The kinetics of TraNS opening in cells is expected to depend on both the number of TraNS on cell membrane and the expression level of target mRNA.

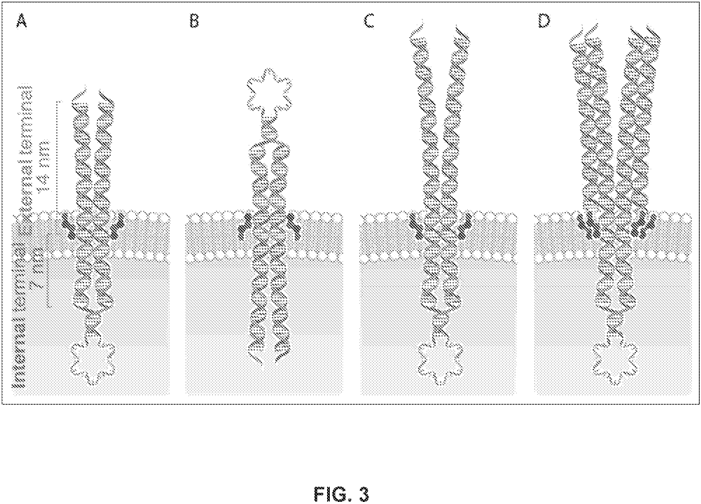

Optimization of TraNS insertion into HEK293 GFP cells. To detect cytosolic RNA molecules, the insertion of TraNS to cell membranes have to result in the correct TraNS orientation (FIG. 3A). For membrane insertion, we conjugated cholesterol molecules (purple; FIGS. 1A and 2A) to TraNS to create a hydrophobic belt around it, as previously-demonstrated for membrane-protein-mimetic DNA nanostructures^{13-15,36}. In the current prototype (FIG. 3A), the cholesterol molecules are placed such that the extramembrane part is ≳2× as large compared to the intramembrane part as shown in (FIGS. 1A-B) for preferential orientation of the TraNS sensors in the membrane. FIGS. 2(A-B) suggests that a significant fraction of TraNS is inserted into liposomes with the correct orientation. Based on a thermodynamics argument, the relative length of the intracellular (IT; FIG. 3A) and extracellular termini (ET; FIG. 3A) of TraNS determines the orientation of the inserted sensors. Specifically, we will systematically vary the IT/ET ratio to optimize the insertion of TraNS into SUVs and into patient-derived exosomes (FIGS. 3A-B). We will compare the kinetics of TraNS opening of different designs. Higher fraction of TraNS with the correct orientation gives faster kinetics of TraNS opening since the molecular beacon of misoriented TraNS is in the opposite side of the cell membranes and is not accessible by the cytosolic RNA molecules (FIG. 3B). We will also test additional modifications, such as extending the length of ET (FIG. 3C) and adding DNA helices to ET (FIG. 3D) to increase the free energy difference between the correct and incorrect orientations of TraNS. Further, we will systematically study the impact of the number and positions of cholesterol molecules to the preferred orientation and performance of TraNS.

### Develop Amplification Reaction and Allosteric TraNS

To achieve adequate signal for FACS, flow cytometry, low-resolution fluorescence microscopy, and other assays, the signal from a conformational change of a single TraNS will be amplified by interfacing TraNS with a programmable Hybridization Chain Reaction (HCR)^{42,43}. Existing work in live-cell genotyping, such as Spherical Nucleic Acid-based SmartFlare^{44,45} (a discontinued product by MilliporeSigma), essentially are gold nanoparticles conjugated to multiple copies of a dsDNA, in which one strand (the “reporter strand”) bears a fluorophore that is quenched by its proximity to the gold core. The fluorescence of the existing techniques is quenched until mRNA binding of the probe relaxes the hairpin and restores the fluorescence. This product failed to meet expectation and discontinued for two reasons - (i) the nanoparticles were hard to reliably deliver into cytosol without transfection agents, (ii) a fraction of the probes do not escape endosomes, do not detect cytosolic mRNAs and produce false positives due to the acidic environment of the endosomes and endosomal DNAse II digestion⁴⁶⁻⁴⁸, and (iii) there was no amplification of signal. The proposed TraNS simultaneously operate in cytoplasm and extracellular medium. Unlike SmartFlare, TraNS bypasses the need for probe uptake by endocytosis and endosomal escape. Moreover, the extracellular terminal of TraNS can be further engineered to interface with DNA signal amplification reactions^{42,43,49,50}.

Preliminary results: Inhibition of HCR by partially-double-stranded initiator molecule. FIG. 4A depicts a schematic illustration of HCR for linear amplification of an initiator molecule. The initiator (a*b*) triggers the opening of the hairpin H1. This reaction will then fuel the opening of the second hairpin. H2 binds cb* sub-section of the first hairpin, and opens up, thus commencing a cyclical strand-displacement process. This continues until all the H1 and H2 hairpins in solution have been exhausted (FIGS. 4B-C).^{42,43} Typical HCR reactions finish in less than 1 hour. In the absence of the initiator DNA, the hairpins are stable (FIG. 4C; left lane). Covering 10-nt segment of the initiator (FIG. 4B) inhibits HCR reactions. The faint smear in the right lane of FIG. 4C shows the false positive leakage of the first prototype of the inhibition strategy is low.

In situ signal amplification for membrane-associated, allosteric TraNS. Inspired by mechanosensitive multi-domain proteins, such as Vinculin, we will couple the OFF→ON conformational change of TraNS with a deprotection reaction of an initiator domain (FIG. 5; teal). Upon binding with a target RNA molecule in the cytosol, a TraNS opens and expose the previously-protected initiator (FIG. 5D) at the extracellular terminal of TraNS, analogous to how mechanical tension exposes the cryptic site of Vinculin (FIGS. 5A-B). The newly-exposed initiator (FIG. 5E) triggers HCR reaction resulting in a linear chain of fluorescent double stranded DNA (FIG. 5E).

We will first evaluate the performance of the allosteric TraNS (FIG. 5C) in HEX293 GFP cell lines. In theory, the binding kinetics of the molecular beacon domain of TraNS and target RNA is expected to be unaffected by the modification at the opposite terminal of TraNS. After assaying the kinetics of the allosteric TraNS, we will incubate the cells-TraNS complex and Cy5-labeled HCR hairpins for 0-3 hours in 1× PBS and 10 mM MgCl₂. Typical HCR reactions finish in less than 1 hour. The progress of the HCR amplification reaction at different time points will be quantified using single-molecule fluorescence microscope with 647 nm excitation wavelength.

Enzymatic removal of sensors. To minimize any perturbation due to TraNS and HCR hairpins, we will develop gentle, enzymatic TraNS removal strategy, leaving the cell with only the intracellular termini of TraNS. We will incubate the HEX293-TraNS-HCR samples with nonspecific Exonuclease V (RecBCD; NEB) or DNase I endonuclease (NEB) for 1 hour to digest the extracellular termini of TraNS and HCR hairpins. The removal of the extracellular termini of TraNS and HCR hairpins will be assayed using single-molecule microscope and flow cytometry.

### Develop FACS Strategy for Isolating Cells Based on Cytosolic mRNAs

Existing cell separation technology target the presence of surface protein markers for antibody binding. Despite their usefulness as biomarkers, one of the drawbacks of surface markers is their functions are often uncharacterized. In many biological samples, cytosolic markers with known functions inform the cell functional types. Without cell lysis, these cytosolic proteins are “untouchable” by antibodies. The proposed TraNS will be specific to mRNA biomarkers (Aim 1) and HCR reaction will amplify the signal (Aim 2). We propose to use the fluorescence intensity of the cells-TraNS for sorting live cells based on their gene expression into functionally different populations.

FACS. HEK293 GFP cells with Cy5-labaled HCR hairpins will be used for the FACS experiments. Flow cytometry and FACS will be performed using a BD FACS Aria IIu Cell Sorter and the data will be analyzed using FlowJo data analysis software. The two different gates, namely TraNS^{high} and TraNS^{low}, will be established based on the emission of the Cy5-labeled HCR hairpins. To validate TraNS-based sorting strategy, we will repeat the FACS experiment based on the GFP emission of HEK293 GFP. The sorted cells will be collected in PBS with 1% FCS for Real Time PCR (RT PCR) analysis, microscopy assay, and cell viability analysis.

RT PCR. Post FACS, sorted cells and negative flowthrough will be spun down and lysed on ice with RLT lysis buffer and 1% DTT. RNA will be purified using RNeasy Micro Kit (Qiagen) and reverse transcribed into cDNA. RT PCR will be performed using on an Eppendorf quantitative real-time PCR thermocyclers. The data will be normalized to the housekeeping gene GAPDH. The correlation between the two FACS methods will be analyzed using home-built Mathematica code.

### References

1 Heather, J. M. & Chain, B. The sequence of sequencers: The history of sequencing DNA. Genomics 107, 1-8, doi:10.1016/j.ygeno.2015.11.003 (2016).

2 Jinek, M. et al. A programmable dual-RNA-guided DNA endonuclease in adaptive bacterial immunity. Science 337, 816-821, doi:10.1126/science.1225829 (2012).

3 Cox, D. B., Platt, R. J. & Zhang, F. Therapeutic genome editing: prospects and challenges. Nat Med 21, 121-131, doi:10.1038/nm3793 (2015).

4 Gaj, T., Gersbach, C. A. & Barbas, C. F., 3rd. ZFN, TALEN, and CRISPR/Cas-based methods for genome engineering. Trends Biotechnol 31, 397-405, doi:10.1016/j.tibtech.2013.04.004 (2013).

5 Alkan, C., Coe, B. P. & Eichler, E. E. Genome structural variation discovery and genotyping. Nat Rev Genet 12, 363-376, doi:10.1038/nrg2958 (2011).

6 Saiki, R. K. et al. Enzymatic amplification of beta-globin genomic sequences and restriction site analysis for diagnosis of sickle cell anemia. Science 230, 1350-1354, doi:10.1126/science.2999980 (1985).

7 Vos, P. et al. AFLP: a new technique for DNA fingerprinting. Nucleic Acids Res 23, 4407-4414, doi:10.1093/nar/23.21.4407 (1995).

8 Saiki, R. K. et al. Primer-directed enzymatic amplification of DNA with a thermostable DNA polymerase. Science 239, 487-491, doi:10.1126/science.2448875 (1988).

9 Tang, F. et al. mRNA-Seq whole-transcriptome analysis of a single cell. Nat Methods 6, 377-382, doi:10.1038/nmeth.1315 (2009).

10 Chen, J. & Seeman, N. C. Synthesis from DNA of a molecule with the connectivity of a cube. Nature 350, 631-633, doi:10.1038/350631a0 (1991).

11 Rothemund, P. W. K. Folding DNA to create nanoscale shapes and patterns. Nature 440, 297-302, doi:10.1038/nature04586 (2006).

12 Douglas, S. M. et al. Self-assembly of DNA into nanoscale three-dimensional shapes. Nature 459, 414-418, doi:10.1038/nature08016 (2009).

13 Burns, J. R., Seifert, A., Fertig, N. & Howorka, S. A biomimetic DNA-based channel for the ligand-controlled transport of charged molecular cargo across a biological membrane. Nat Nanotechnol 11, 152-156, doi:10.1038/nnano.2015.279 (2016).

14 Krishnan, S. et al. Molecular transport through large-diameter DNA nanopores. Nat Commun 7, 12787, doi:10.1038/ncomms12787 (2016).

15 Langecker, M. et al. Synthetic Lipid Membrane Channels Formed by Designed DNA Nanostructures. Science 338, 932-936, doi:10.1126/science.1225624 (2012).

16 Göpfrich, K. et al. Ion Channels Made from a Single Membrane-Spanning DNA Duplex. Nano Letters 16, 4665-4669, doi:10.1021/acs.nanolett.6b02039 (2016).

17 Thomsen, R. P. et al. A large size-selective DNA nanopore with sensing applications. Nature Communications 10, doi:10.1038/s41467-019-13284-1 (2019).

18 Diederichs, T. et al. Synthetic protein-conductive membrane nanopores built with DNA. Nature Communications 10, doi:10.1038/s41467-019-12639-y (2019).

19 List, J., Weber, M. & Simmel, F. C. Hydrophobic Actuation of a DNA Origami Bilayer Structure. Angewandte Chemie International Edition 53, 4236-4239, doi:10.1002/anie.201310259 (2014).

20 Ohmann, A. et al. A synthetic enzyme built from DNA flips 107 lipids per second in biological membranes. Nature Communications 9, doi:10.1038/s41467-018-04821-5 (2018).

21 Walter, H.-K. et al. “DNA Origami Traffic Lights” with a Split Aptamer Sensor for a Bicolor Fluorescence Readout. Nano Letters 17, 2467-2472, doi:10.1021/acs.nanolett.7b00159 (2017).

22 Kuzyk, A. et al. Reconfigurable 3D plasmonic metamolecules. Nature Materials 13, 862-866, doi:10.1038/nmat4031 (2014).

23 Liu, M. et al. A DNA tweezer-actuated enzyme nanoreactor. Nature Communications 4, doi:10.1038/ncomms3127 (2013).

24 Funke, J. J. & Dietz, H. Placing molecules with Bohr radius resolution using DNA origami. Nat Nanotechnol 11, 47-52, doi:10.1038/nnano.2015.240 (2015).

25 Zhou, C., Yang, Z. & Liu, D. Reversible Regulation of Protein Binding Affinity by a DNA Machine. Journal of the American Chemical Society 134, 1416-1418, doi:10.1021/ja209590u (2012).

26 Manglik, A. & Kobilka, B. The role of protein dynamics in GPCR function: insights from the beta2AR and rhodopsin. Curr Opin Cell Biol 27, 136-143, doi:10.1016/j.ceb.2014.01.008 (2014).

27 Lister, F. G. A., Le Bailly, B. A. F., Webb, S. J. & Clayden, J. Ligand-modulated conformational switching in a fully synthetic membrane-bound receptor. Nat Chem 9, 420-425, doi:10.1038/Nchem.2736 (2017).

28 Venkatakrishnan, A. J. et al. Molecular signatures of G-protein-coupled receptors. Nature 494, 185-194, doi:10.1038/naturell896 (2013).

29 Liu, Q. et al. Circulating exosomal microRNAs as prognostic biomarkers for non-small-cell lung cancer. Oncotarget 8, doi:10.18632/oncotarget.14369 (2016).

30 Zheng, W. et al. MicroRNA-21: A promising biomarker for the prognosis and diagnosis of non-small cell lung cancer (Review). Oncology Letters, doi:10.3892/ol.2018.8972 (2018).

31 Moriyama, T. et al. MicroRNA-21 modulates biological functions of pancreatic cancer cells including their proliferation, invasion, and chemoresistance. Molecular Cancer Therapeutics 8, 1067-1074, doi:10.1158/1535-7163.Mct-08-0592 (2009).

32 Gu, L. et al. Inhibition of miR-21 induces biological and behavioral alterations in diffuse large B-cell lymphoma. Acta Haematol 130, 87-94, doi:10.1159/000346441 (2013).

33 Medina, P. P., Nolde, M. & Slack, F. J. OncomiR addiction in an in vivo model of microRNA-21-induced pre-B-cell lymphoma. Nature 467, 86-90, doi:10.1038/nature09284 (2010).

34 Venturutti, L. et al. Stat3 regulates ErbB-2 expression and co-opts ErbB-2 nuclear function to induce miR-21 expression, PDCD4 downregulation and breast cancer metastasis. Oncogene 35, 2208-2222, doi:10.1038/onc.2015.281 (2016).

35 Asangani, I. A. etal. MicroRNA-21 (miR-21) post-transcriptionally downregulates tumor suppressor Pdcd4 and stimulates invasion, intravasation and metastasis in colorectal cancer. Oncogene 27, 2128-2136, doi:10.1038/sj.onc.1210856 (2008).

36 Langecker, M., Arnaut, V., List, J. & Simmel, F. C. DNA Nanostructures Interacting with Lipid Bilayer Membranes. Accounts Chem Res 47, 1807-1815, doi:10.1021/ar500051r (2014).

37 Fareh, M. et al. TRBP ensures efficient Dicer processing of precursor microRNA in RNA-crowded environments. Nat Commun 7, 13694, doi:10.1038/ncomms13694 (2016).

38 Cheng, H. T., Megha & London, E. Preparation and properties of asymmetric vesicles that mimic cell membranes: effect upon lipid raft formation and transmembrane helix orientation. J Biol Chem 284, 6079-6092, doi:10.1074/jbc.M806077200 (2009).

39 Conway, J. W., McLaughlin, C. K., Castor, K. J. & Sleiman, H. DNA nanostructure serum stability: greater than the sum of its parts. Chemical Communications 49, doi:10.1039/c2cc37556g (2013).

40 Sago, C. D. et al. Barcoding chemical modifications into nucleic acids improves drug stability in vivo. Journal of Materials Chemistry B 6, 7197-7203, doi:10.1039/c8tb01642a (2018).

41 Gerling, T., Kube, M., Kick, B. & Dietz, H. Sequence-programmable covalent bonding of designed DNA assemblies. Science Advances 4, doi:10.1126/sciadv.aau1157 (2018).

42 Dirks, R. M. & Pierce, N. A. Triggered amplification by hybridization chain reaction. Proc Natl Acad Sci USA 101, 15275-15278, doi:10.1073/pnas.0407024101 (2004).

43 Choi, H. M. T. et al. Third-generation in-situ hybridization chain reaction: Multiplexed, quantitative, sensitive, versatile, robust. Development 145, doi:10.1242/dev.165753 (2018).

44 Seferos, D. S., Giljohann, D. A., Hill, H. D., Prigodich, A. E. & Mirkin, C. A. Nanoflares: probes for transfection and mRNA detection in living cells. J Am Chem Soc 129, 15477-15479, doi:10.1021/ja0776529 (2007).

45 Prigodich, A. E. et al. Multiplexed nanoflares: mRNA detection in live cells. Anal Chem 84, 2062-2066, doi:10.1021/ac202648w (2012).

46 Choi, C. H., Hao, L., Narayan, S. P., Auyeung, E. & Mirkin, C. A. Mechanism for the endocytosis of spherical nucleic acid nanoparticle conjugates. Proc Natl Acad Sci USA 110, 7625-7630, doi:10.1073/pnas.1305804110 (2013).

47 Czarnek, M. & Bereta, J. SmartFlares fail to reflect their target transcripts levels. Sci Rep 7, 11682, doi:10.1038/s41598-017-11067-6 (2017).

48 Budik, S., Tschulenk, W., Kummer, S., Walter, I. & Aurich, C. Evaluation of SmartFlare probe applicability for verification of RNAs in early equine conceptuses, equine dermal fibroblast cells and trophoblastic vesicles. Reprod Fertil Dev 29, 2157-2167, doi:10.1071/RD16362 (2017).

49 Kishi, J. Y., Schaus, T. E., Gopalkrishnan, N., Xuan, F. & Yin, P. Programmable autonomous synthesis of single-stranded DNA. Nat Chem 10, 155-164, doi:10.1038/nchem.2872 (2018).

50 Ali, M. M. et al. Rolling circle amplification: a versatile tool for chemical biology, materials science and medicine. Chem Soc Rev 43, 3324-3341, doi:10.1039/c3cs06439j (2014)

We claim:

1. A cell detection system comprising:
(i) a transmembrane nanosensor comprising a lipid-conjugated DNA comprising a first hairpin stem-loop comprising:
a loop comprising a polynucleotide sequence which is complementary to a target polynucleotide,
a stem comprising complementary 5′ and 3′ domains,
a fluorophore,
a quencher paired to the fluorophore, and
a masked hybridization chain reaction (HCR) initiator domain; and
(ii) components for HCR comprising: a set of metastable hairpins configured for HCR, wherein at least one of the metastable hairpins hybridizes to the HCR initiator domain of the nanosensor and initiates an HCR reaction when the HCR initiator is unmasked;
wherein upon the first hairpin stem-loop binding to the target polynucleotide, the nanosensor transitions from a closed conformation to an open conformation exposing the HCR initiator domain and allowing for fluorescence the fluorophore without quenching.

2. The transmembrane nanosensor of the system of claim 1, wherein the lipid-conjugated DNA spans a lipid bilayer.

3. The transmembrane nanosensor of the system of claim 2, wherein the lipid bilayer is a cellular outer membrane or episome.

4. The transmembrane nanosensor of the system of claim 1, wherein the target polynucleotide is an RNA or a DNA.

5. The transmembrane nanosensor of the system of claim 1, wherein the target polynucleotide is a messenger RNA (mRNA) or microRNA (miRNA).

6. The transmembrane nanosensor of the system of claim 1, wherein the lipid comprises a cholesterol molecule.

7. The system of claim 1, wherein the metastable hairpins configured for HCR comprise a detectable label.

8. The system of claim 7, wherein the metastable hairpins configured for HCR comprises a quencher.

9. A method of labeling a cell comprising a target polynucleotide, the method comprising:
(a) contacting the cell with the system of claim 1, and
(b) detecting fluorescence of the transmembrane nanosensor; whereby fluorescence of the transmembrane nanosensor is indicative of the cell comprising the target polynucleotide, and/or
(c) detecting an HCR product, whereby detection of the HCR product is indicative of the cell comprising the target polynucleotide.

10. The method of claim 9, further comprising separating the detected cell away from another cell.

11. The method of claim 10, wherein separating comprises fluorescent activated cell sorting (FACS).

12. The method of claim 10, wherein separating comprises capturing the HCR onto a solid support.

13. A method of diagnosing a disease in a subject, the method comprising:
(a) contacting a cell derived from the subject with the system according to claim 1; and
(b) detecting a fluorescence of the transmembrane nanosensor, whereby the fluorescence of the nanosensor indicates the presence of a target polynucleotide indicative of the disease in the subject, and/or
(c) detecting the presence of an HCR product, whereby the presence of the HCR product indicates the presence of a target polynucleotide indicative of the disease in the subject.

14. The method of claim 13, wherein the cell is from a liquid biological sample from the subject.

15. The method of claim 13, wherein the target polynucleotide is an miRNA, mRNA or DNA biomarker specific to cancer.

16. The method of claim 13, wherein the detecting a fluorescence comprises quantitating the fluorescence.
